(19) United States
(12) Patent Application Publication
(10) Pub. No.: US 2024/0394885 A1
(43) Pub. Date: Nov. 28, 2024

(54) AUTOMATIC OPTIMIZATION OF PARAMETERS OF AN IMAGE PROCESSING CHAIN

(71) Applicant: Siemens Healthineers AG, Forchheim (DE)

(72) Inventors: Dominik ECKERT, Fuerth (DE); Ludwig RITSCHL, Buttenheim (DE); Steffen KAPPLER, Hallerndorf-Pautzfeld (DE)

(73) Assignee: Siemens Healthineers AG, Forchheim (DE)

(21) Appl. No.: 18/674,031

(22) Filed: May 24, 2024

(30) Foreign Application Priority Data
May 26, 2023 (EP) 23175672.7

(51) Int. Cl. G06T 7/00

(52) U.S. Cl. CPC G06T 7/0014; G06T 2207/30068; G06T 2207/30168

(57) ABSTRACT

A method for AI-assisted generating an adapted medical image processing chain comprises receiving medical image data representing only a piece of information of a complete matching pair of medical image data related to a target flavor, wherein a missing piece of information of the complete matching pair is missing. The method also comprises generating an estimated medical image by applying a medical image processing chain to a raw projection medical image that is related to the received medical image data. Further, the method comprises determining a result of a comparison based on the estimated medical image and a target medical image which is also related to the received medical image data. The method furthermore comprises generating an adapted medical image processing chain by adapting the medical image processing chain based on the result of the comparison. The missing piece of information is generated based on the received medical image data.

### CROSS-REFERENCE TO RELATED APPLICATION(S)

The present application claims priority under 35 U.S.C. § 119 to European Patent Application No. 23175672.7, filed May 26, 2023, the entire contents of which is incorporated herein by reference.

### FIELD

One or more example embodiments of the present invention relates to a method for AI-assisted generating an adapted medical image processing chain. Further, one or more example embodiments of the present invention relates to a method for generating a trained AI-based model for estimating a missing piece of information of a complete matching pair of medical image data related to a target flavour. Furthermore, one or more example embodiments of the present invention concerns an adaption device.

### RELATED ART

X-rays have different image impressions called flavors. These flavors depend on the hardware used to record them as well as on the operation principle and the parameter set of the applied reconstruction and the processing algorithm. The influence of the chosen recording system on the X-ray image impression can be observed in FIG. 1 and FIG. 2. FIG. 1 shows an example of two reconstructed X-ray images of the same breast, but recorded with a different parameter set of the post processing algorithm. The same physical phantom scanned with two different X-ray machines generating medical image with different flavors, i.e. impressions, is presented in FIG. 2.

Radiologists are used to a particular flavor. Hence, changing or working with different image impression leads to an increase in the reading time and a decrease of the reliability of the diagnosis. This is especially problematic, if different X-ray images with different flavors must be compared against each other. However, there are several reasons, which make the use or exchange of various x-ray systems necessary: An X-ray system exchange or update with more recent technology increases the image quality and consequently the reliability of the diagnosis, an old X-ray machine must be replaced due to a defect, or a radiologist changes the working side.

When a new system or software version is installed at a customer site, service or image quality specialists adjust post processing parameters by hand together with the customer, using some example cases from the local PACS (PACS is an acronym for Picture Archiving and Communication System). This is very time consuming, and the results are not always satisfying.

For adapting an image processing chain, a pair of images is necessary, wherein a first image comprises an unprocessed raw projection medical image and a second image comprises a processed target medical image including the desired flavor and both types of images depict the same image content. However, typically, there is only one set of processed images from the radiologist with the desired flavor, but no matching unprocessed raw projection medical image. The absence of such a complete matching pair prevents the adaption of an image processing chain in which raw projections are fed into the processing chain and in which the so obtained processed medical images are compared against their corresponding matching target medical images comprising the desired flavor.

### SUMMARY

Hence, a general problem is to adapt characteristics of processed X-ray images to desired image characteristics, in particular the desired flavor.

This problem is solved by a method for AI-assisted generating an adapted medical image processing chain according to claim 1, by a method for generating a trained AI-based model for estimating a missing piece of information of a complete matching pair of medical image data related to a target flavor according to claim 10 and by an adaption device according to claim 13.

### BRIEF DESCRIPTION OF THE DRAWINGS

One or more example embodiments of the present invention is explained below with reference to the figures enclosed once again. The same components are provided with identical reference numbers in the various figures.

The figures are usually not to scale.

FIG. 1 shows a schematic view on two reconstructed volumes of the same breast scan showing two different flavors,

FIG. 2 shows a schematic view on a scan of the same phantom generated by different medical imaging systems,

FIG. 3 shows a schematic diagram illustrating a method for AI-assisted generating an adapted medical image processing chain according to a first embodiment of the invention,

FIG. 4 shows a schematic diagram illustrating a method for AI-assisted generating an adapted medical image processing chain according to a second embodiment of the invention,

FIG. 5 shows a flow chart diagram illustrating the method for AI-assisted generating an adapted medical image processing chain according to the first embodiment of the invention,

FIG. 6 shows a flow chart diagram illustrating the method for AI-assisted generating an adapted medical image processing chain according to the second embodiment of the invention,

FIG. 7 shows a flow chart diagram illustrating the method for generating a trained AI-based model for estimating a missing piece of information of a complete matching pair of medical image data related to a target flavor, in particular synthetic image data or representation data, according to an embodiment of the invention, and

FIG. 8 shows a schematic view on an adaption device according to an embodiment of the invention.

### DETAILED DESCRIPTION

By the method for AI-assisted generating an adapted medical image processing chain (AI is an acronym for “artificial intelligence”), medical image data representing only a piece of information of a complete matching pair of medical image data related to a target flavor are received, wherein another piece of information of the complete matching pair is missing. A complete matching pair comprises a pair of a raw projection medical image and a target medical image. A complete matching pair of medical image data means that both images of the “matching pair” include the same image content and the target medical image includes the target flavor. A piece of information means that the received medical image data are incomplete and do not represent the ideal complete matching pair. Hence, there is a lack of information at the beginning of the method according to one or more example embodiments of the present invention, which has to be compensated for achieving a correct adaption of the medical image processing chain to a target flavor.

As later described in detail, an “incomplete matching pair” preferably means that the situation may arise that only one of the following sets of images are available:
only a raw projection medical image,
only a target medical image,
a raw projection medical image and a target medical image, wherein the content of the raw projection medical image is not the same as the content of the target medical image.

Based on the available image data measurements have to be performed to compensate the lack of information. As later described, in one variant, an AI-based model compensates the lack of information about a target flavor, in another variant, an AI-based model compensates the lack of information about raw projection medical image data corresponding to the target medical image and in a third variant, an AI-based model compensates the lack of paired medical images with the same content.

Firstly, an estimated medical image is automatically generated by applying the medical image processing chain to a raw projection medical image. A medical image processing chain comprises a sequence of filter units for processing a raw projection medical image. The characteristics of each of the filter units can be modified by altering parameter values of parameters assigned to each of these filter units. Parameters of an image chain relate to the windowing, which is related to the division of grey value intervals. Further, parameters can also be related to the presentation of the background. Furthermore, parameters can also concern cutting or resampling of value ranges, the use of color look-up tables or filters, and the adjustment of the bit depth or sampling depth.

A raw projection medical image is an unprocessed medical image as it directly results from the X-ray projection and image recordation without any post-processing.

For the object of adapting the resulting flavor of an image processing chain to a desired type of flavor, an image processing chain has to be adapted. For that object, the medical image processing chain is applied to the raw projection medical image. A flavor can be defined by a set of parameters. Such a set of parameters preferably comprises at least one of the following types of parameters: the sharpness of the image in high frequencies, low contrasts, dynamic behaviour, in particular windowing for specific organs like abdomen or lung, dynamic range compression, tone mapping.

Tone mapping, tone reproduction or dynamic range compression are synonymous terms that describe the compression of the dynamic range of high-contrast images (high dynamic range images), i.e. digital images with a high brightness range. With tone mapping, the contrast range of a high-contrast image is reduced in order to be able to display it on conventional output devices.

Further, a result of a comparison based on the estimated medical image and a target medical image is automatically determined. For the comparison and adaption, different actions have to be taken to compensate the lack of information.

In particular, for comparison the style of different images, different options appear. For instance, local Laplacian filters exist for transferring the style from one image to another. These filters have originally been investigated for photographical images and are for example part of a photo editing software. These filters can be modified in a way that the remap functions of each layer of the Laplacian pyramid reflect the style difference of two images: The closer the remap functions are on the diagonal, the greater the similarity between the two medical images is. This can be reflected by one number using the mean difference of the areas under curve. Another possibility would be to analyze the histograms of different frequency bands of the two medical images. For instance, a histogram matching technique was introduced to standardize image impressions as preprocessing technique for increasing the robustness of machine learning algorithms.

Accordingly, a measure of similarity can be derived by comparing the frequency-band histograms (e.g., mean and standard deviation) of the two medical images.

As later discussed in detail, the type of comparison depends on the type of the medical image data available for the comparison.

Based on the result of the comparison, an adapted medical image processing chain is generated by adapting the medical image processing chain. The adaption based on the comparison, i.e. the similarity measure, is preferably used within an optimization procedure, e.g. a gradient descent method or a

Newton method for an iterative adjustment of parameters of the medical image processing chain.

Advantageously, a medical image processing chain is automatically adapted for generating medical image data with a desired flavour without involving a human expert having to intervene in the adaption process based on an incomplete matching pair of medical images. By adapting a medical image processing chain of a new medical imaging system, in particular an X-ray imaging system, to a flavor of an old medical imaging system used by radiologists before, a more precise and faster reading of the medical images on the new medical imaging system by the radiologists is achieved.

As later discussed in detail, for compensating a lack of an available complete matching pair of a raw projection medical image and a target medical image, wherein both include the same image content, the missed piece of information is compensated or even generated using a trained AI-based model. The trained AI-based model either directly generates the missed medical images or generates other types of data being appropriate for a comparison for adapting the medical image processing chain.

For that object, a method for generating a trained AI-based model for estimating a missing piece of information of a complete matching pair of medical image data related to a target flavor, preferably synthetic image data or representation data, according to one or more example embodiments of the present invention is also provided.

The method comprises the steps of:
generating input data, wherein the input data comprise medical image data representing only a piece of information of a complete matching pair related to a target flavor,
applying the input data to an AI-based model to be trained, wherein result data are generated,
training the AI-based model based on the result data,
providing the trained AI-based model.

The medical image data preferably comprise at least one of the following data sets:
a target medical image,
a raw projection medical image,
an estimated medical image.

The result data may comprise information useful for achieving the missing piece of information, preferably synthetic image data or representation data, used for compensating the missing piece of information of the input data.

Advantageously, an AI-based model used for estimating missing information, preferably synthetic image data or representation data, can be flexibly adapted to a deliberate data basis of training data.

If a supervised training is to be carried out, the method for generating a trained AI-based model for estimating missing information of a piece of information of a complete matching pair of medical image data related to a target flavor, preferably synthetic image data or representation data, according to one or more example embodiments of the present invention, comprises the step of generating labelled input data including input data and validated result data. The labelled input data comprise only a piece of information of a complete matching pair of medical image data related to a target flavor and the validated result data comprise the missing information of the piece of information of a complete matching pair of medical image data related to a target flavor.

Preferably, the labelled input data comprise one of the following data sets:
a target medical image as input data and a validated raw projection medical image as validated result data,
a raw projection medical image as input data and a validated target medical image as validated result data,
an estimated medical image or a target medical image as input data and representation data representing a flavor of the medical image as validated result data.

The above mentioned variant further comprises the steps of:
applying the labelled input data to an AI-based model to be trained, wherein result data are generated,
training the AI-based model based on the result data and the validated result data,
providing the trained AI-based model.

As validated result data, a medical image comprising a predetermined flavor of the assigned medical image or representation data representing the flavor of the assigned medical image are to be understood. The training of the AI-based model can be implemented using a backpropagation algorithm or a cost function for adapting the AI-based model to the labelled input data. Using a supervised training comprises the advantage that the training data basis need not to be as extensive as it has to be in the variant of an unsupervised or self-supervised training. Further, the artificial neural network structure need not to be as complex as for the variant of an unsupervised or self-supervised training.

Alternatively, the method for generating a trained AI-based model for estimating a missing piece of information, preferably synthetic image data or representation data, according to one or more example embodiments of the present invention, can be implemented as a training for an unsupervised or self-supervised network, in particular a generative adversarial network. That means that the training data need not to be labelled at al. Hence, in the alternative variant, the training is performed by using unlabelled input data and the step of training the AI-based model comprises an unsupervised or self-supervised training step. Such a variant involves less effort compared to a supervised training using labelled training data.

A generative adversarial network consists of two artificial neural networks performing a zero-sum game. One of them creates candidates (the generator), the second neural network evaluates the candidates (the discriminator).

Typically, the generator maps from a vector of latent variables to the desired result space. For example, the latent variables inform about the style of a target medical image. The aim of the generator is to learn how to generate results according to a specific distribution. For example, the results comprise the synthetic raw projection medical image. The discriminator, on the other hand, is trained to distinguish the results of the generator from the data from the real, given distribution. The generator's objective function or cost function is then to produce results that the discriminator cannot distinguish. This should gradually adjust the generated distribution to the real distribution.

For that reason, a sufficiently large data set consisting of real unlabelled input data is required to train such a model. These unlabelled input data may comprise incomplete matching pairs of medical image data related to a target flavor and preferably data sets including a target medical image as input data and data sets including a raw projection medical image as input data.

These input data are used to train a discriminator until it reaches an acceptable level of accuracy. During subsequent training, a generator is given a random sample of a distribution of result data chosen from a previously defined range of latent variables. From this, the generator tries to generate a new distribution. This distribution is then presented to the discriminator, which tries to distinguish it from a real one. The weights of both models are independently improved by backpropagation, allowing the generator to create better distributions and the discriminator to better recognize them. Through this game, both models constantly improve each other, which, given sufficient training time, leads to generated distributions that cannot be distinguished from real ones.

The adaption device according to one or more example embodiments of the present invention comprises an input interface for receiving medical image data representing a piece of information of a complete matching pair related to a target flavor. The adaption device also comprises an estimation unit for generating an estimated medical image by applying a medical image processing chain to a raw projection medical image. Further, the adaption device according to one or more example embodiments of the present invention includes a comparison unit for determining a result of a comparison based on the estimated medical image and a target medical image. Furthermore, the adaption device according to one or more example embodiments of the present invention comprises an adaption unit for generating an adapted medical image processing chain by adapting the medical image processing chain based on the result of the comparison.

The adaption device according to one or more example embodiments of the present invention shares the advantages of the method for AI-assisted generating an adapted medical image processing chain according to one or more example embodiments of the present invention.

Some units or modules of the adaption device mentioned above, in particular the estimation unit, the comparison unit and the adaption unit, can be completely or partially realized as software modules running on a processor of a respective computing system, e.g. of a control device of a finding system or a medical imaging system. A realization largely in the form of software modules can have the advantage that applications already installed on an existing computing system can be updated, with relatively little effort, to install and run these units of the present application. The object of one or more example embodiments of the present invention is also achieved by a computer program product with a computer program or by a computer program that is directly loadable into the memory of a computing system, and which comprises program units to perform the steps of the inventive method for AI-assisted generating an adapted medical image processing chain and the steps of the method for generating a trained AI-based model for estimating synthetic image data or representation data, when the program is executed by the computing system.

In particular, the method for AI-assisted generating an adapted medical image processing chain may include the following steps executable by a computer program: the step of generating an estimated medical image, the step of determining a result of a comparison based on the estimated medical image and a target medical image and the step of generating an adapted medical image processing chain.

The method for AI-assisted generating an adapted medical image processing chain may also include the following steps executable by a computer program later described in detail: the step of generating a synthetic target medical image by applying a first trained AI-based model to the raw projection medical image or a synthetic raw projection medical image by applying a second trained AI-based model to the target medical image or first representation data representing a flavor of the target medical image by applying a third trained AI-based model to the target medical image.

The method for AI-assisted generating an adapted medical image processing chain may also include the following steps executable by a computer program later described in detail: the step of generating an estimated medical image by applying the medical image processing chain to the raw projection medical image or the synthetic raw projection medical image, the step of determining a result of a comparison between the estimated medical image and the target medical image or the synthetic target medical image or generating second representation data representing a flavor of the estimated medical image by applying the third trained AI-based model to the estimated medical image and determining a result of a comparison between the first representation data and the second representation data and the step of generating an adapted modelled medical image processing chain by adapting the medical image processing chain based on the result of the comparison.

Further, the method for generating a trained AI-based model for estimating synthetic image data or representation data may also include the following steps executable by a computer program: the step of generating input data, the step of applying the input data to an AI-based model to be trained, wherein result data are generated, and the step of training the AI-based model based on the result data.

In addition to the computer program, such a computer program product can also comprise further parts such as documentation and/or additional components, also hardware components such as a hardware key (dongle etc.) to facilitate access to the software.

A computer readable medium such as a memory stick, a hard-disk or other transportable or permanently-installed carrier can serve to transport and/or to store the executable parts of the computer program product so that these can be read from a processor unit of a computing system. A processor unit can comprise one or more microprocessors or their equivalents.

The dependent claims and the following description each contain particularly advantageous embodiments and developments. In particular, the claims of one claim category can also be further developed analogously to the dependent claims of another claim category. In addition, within the scope of the invention, the various features of different exemplary embodiments and claims can also be combined to form new exemplary embodiments.

In a variant of the method for AI-assisted generating an adapted medical image processing chain according to one or more example embodiments of the present invention, the automatic steps are automatically iteratively repeated using the adapted medical image processing chain of the third step as medical image processing chain in the first step. Advantageously, the adaption is improved with every iteration step such that an iterative approach to an optimized parameterization of the medical image processing chain is achieved. The adaption of the medical image processing chain based on a comparison based on the estimated medical image and the target medical image can be formulated by using an objective function, also called a loss or an error function, which indicates if the desired flavor is met. The parameter values of the parameters of the medical image processing chain are iteratively adjusted until the value of the objective function is as low as possible.

Preferably, the three steps of the method for AI-assisted generating an adapted medical image processing chain are automatically iteratively repeated, until a predetermined quality criteria or optimum criteria for the result of the comparison in the second step is achieved. Advantageously, the iteration is repeated, until the precision of the optimization achieves a predetermined level or an optimum level. Ideally, the medical image processing chain is differentiable and the parameter values of the parameters medical image processing chain can be adjusted by doing a stochastic gradient descent. Otherwise, an optimization can be performed based on numerical approaches.

In a variant of the method for AI-assisted generating an adapted medical image processing chain, wherein a matching pair of a raw projection medical image and a target medical image with the same content is missed and only a raw projection medical image is received and therefore available, a synthetic target medical image is generated by applying a first trained AI-based model to the raw projection medical image in advance. Further, the comparison in the second step is performed by comparing the estimated medical image with the synthetic target medical image being used as the target medical image. Advantageously, a matching pair of a raw projection medical image and a target medical image is artificially generated. The artificially generated matching pair can be used for adapting a medical image processing chain by comparing these different images including the same content. Advantageously, an adaption of a medical imaging processing chain is possible even if a matching pair of a raw projection medical image and a target medical image with the same content is not available.

In a further variant of the method for AI-assisted generating an adapted medical image processing chain, the received medical image data comprises a target medical image, but not a raw projection medical image. Hence, a synthetic raw projection medical image is generated by applying a second trained AI-based model to the target medical image. Typically, there is only one set of processed images from the radiologist with the desired style, but there are not matching unprocessed raw projection medical image including the same image content. Then, the generation of an estimated medical image in the first step is performed by applying the medical image processing chain to the synthetic raw projection medical image being used as the raw projection medical image and the comparison in second step is performed by comparing the estimated medical image with the target medical image. Advantageously, an adaption of a medical imaging processing chain is possible even if a target medical image of the desired flavor is available, but no matching raw projection medical image is available.

In an alternative variant of the method for AI-assisted generating an adapted medical image processing chain, a pair of an unprocessed raw projection medical image and a target medical image of the desired flavor is received and therefore available, but the content of the raw projection medical image is not the same as the content of the target medical image.

Therefore, first representation data representing a flavor of the target medical image by applying a third trained AI-based model to the target medical image are generated in advance.

Then, in the second step of determining a result, second representation data representing a flavor of the estimated medical image are generated by applying the third trained AI-based model to the estimated medical image and the result is determined by a comparison between the first representation data and the second representation data. Advantageously, the content of the pair of medical image data used for adapting the medical image processing chain does not need to be the same, which improves the flexibility of the adaption process.

Preferably, the first representation data and the second representation data comprise a unit vector and the number of the dimension of the unit vector is the same as the number of different possible flavors represented by the first representation data and the second representation data. Advantageously, a comparison between different representation data can be performed based on a norm of a difference between the two unit vectors representing the first representation data and the second representation data, wherein the result is normalized and therefore easily comparable with a normal.

Preferably, the result comprises a difference between the compared images or compared representation data and the predetermined quality criteria comprises a maximum allowed difference or norm of a difference between the first and second representation data or an indication for a minimized value of the difference or a minimized value of a norm of the difference. Advantageously, the minimum precision of the adaption of the medical image processing chain can be controlled and predetermined.

Particularly preferably, the adapted modelled medical image processing chain is achieved by generating an adapted parameter value by adapting a parameter value of at least one parameter of the medical image processing chain and the adapted medical image processing chain is achieved by taking over the adapted parameter value for the adapted medical image processing chain. Advantageously, the parameter values of the medical image processing chain can be automatically adapted and even a matching set of a pair of a raw projection medical image and a target medical image with the same content is not available.

In a variant of the method for generating an adapted medical image processing chain according to one or more example embodiments of the present invention, the first or second AI-based model is trained using an un- or self-supervised method.

Unsupervised learning refers to machine learning with no known target values and no reward from the environment. The learning algorithm or model tries to recognize patterns in the input data that deviate from structureless noise.

Preferably, the un- or self-supervised method comprises a generative adversarial network. A generative adversarial network (GAN for short) is a machine learning model capable of generating data. It consists of two competing artificial neural networks (ANN for short). One has the task of generating real-looking data, the other classifies the data as real or artificial. Through constant learning and many iteration steps, the generated data is getting better and better. A typical area of application is the creation of realistic-looking artificial images. Advantageously, synthetic raw projection medical images and synthetic target medical images can be generated very realistic. Prominent representatives based on GANs are described in Zhu, Jun-Yan, et al. “Unpaired image-to-image translation using cycle-consistent adversarial networks.” Proceedings of the IEEE international conference on computer vision. 2017 and in Park, Taesung, et al. “Contrastive learning for unpaired image-to-image translation.” European conference on computer vision. Springer, Cham, 2020.

It is also possible to “augment” different flavors with an image processing chain. In that way, there are matching pairs of raw projection medical images and processed target medical images. An artificial neural network can then be trained in an end-to-end fashion to learn to match flavoured target medical images back to raw projection medical images or vice versa. Diffusion models shown in Zhao, Min, et al. “Egsde: Unpaired image-to-image translation via energy-guided stochastic differential equations.” arXiv preprint arXiv: 2207.06635 (2022) might prove especially suitable for this task. If the data augmentation is diverse enough, the network is then able to match unseen flavors from desired flavor back.

It is also possible to compare multiple medical images at once. The average of the differences can then be used to optimize the medical image processing chain. To train such a flavor representing network, a dataset of X-ray medical images with different flavors is necessary. Moreover, the information, which medical images have the same flavor, must be included. However, no matching pairs are needed. The flavor representing network has then to be trained to minimize the representation distance between two medical images of the same flavor and maximize the distance between two medical images with different flavors.

Depending on the number of different flavors, different training strategies are suitable. For a discrete number of flavors, a classification in a one hot encoder fashion is possible. A 1-of-n code, also known as one-hot coding, represents numbers in binary, usually for use in digital technology or computers. In that procedure, each flavor is represented by a unit vector in the direction of only one dimension, e.g. [0, 0, 1, 0]. In that example, we would have four different flavors, for each flavor the vector points in another direction. The network is trained to estimate these vectors. Ideally, unseen flavors match either one of the training flavors or lie between some flavors used for training, so that the represented flavor is still a unit vector and lies on a unit sphere, e.g. [0, 0.5, 0.866, 0].

Due to data augmentation, in that case flavor augmentation, an infinite number of flavors is possible. In that case, another loss function must be applied to train the flavor representing network. Contrastive learning, described in Chen, Ting, et al. “A simple framework for contrastive learning of visual representations.” International conference on machine learning. PMLR, 2020, and siamese learning, described in Melekhov, Iaroslav, Juho Kannala, and Esa Rahtu. “Siamese network features for image matching.” 2016 23rd international conference on pattern recognition (ICPR). IEEE, 2016, are two possible options.

Typically, in contrastive learning, the network get's a set of images for each training iteration. It then must choose which of these images belong together and which images do not belong together. Using the siamese loss, the network only gets two medical images and must decide if these two medical images belong together or not. Usually, both approaches are used, to train a network to represent the content of an image. In our case, the network must find the right flavors, which belong together and so it learns to represent the X-ray image flavor. In a contrastive or a siamese procedure, several activated last layers of the network serve as representations.

Hence, in an also preferred variant of the method for generating an adapted medical image processing chain of one or more example embodiments of the present invention, the representation data are compared and the number of different possible flavors is infinite and the first representation data and the second representation data comprise several activated last layers of an AI-based network which is based on contrastive learning or siamese learning. Advantageously, the scale of different flavors can be arbitrarily finely subdivided.

In a variant of the method for generating a trained AI-based model for estimating synthetic image data or representation data, augmented labelled input data are generated as input data by applying a modelled medical image processing chain to unlabelled input data. Advantageously, the training data basis can be extended to more different training data which increases the flexibility and robustness of the trained AI-based model.

In a preferred variant of the adaption device according to one or more example embodiments of the present invention, the adaption device comprises an input interface for receiving a medical image processing chain and one of the following image data:
a raw projection medical image or
a target medical image or
a raw projection medical image and a target medical image, wherein the content of the raw projection medical image is not the same as the content of the target medical image.

As explained in detail, if a complete matching pair is provided, the adaption of the medical imaging processing chain can be achieved by a direct comparison between an estimated medical image generated based on the raw projection medical image and the target medical image of the matching pair. If one member of the matching pair is missed, the missed member can be generated using an AI-based model. If one member of the matching pair is missed, however a raw projection medical image and a target medical image including a different content are available, instead of a direct comparison, a comparison of the flavor of these images with different content can be performed for generating an adapted medical imaging processing chain for reconstruction of medical images with the desired flavor.

Also preferably, the adaption device according to one or more example embodiments of the present invention comprises a generation unit for generating at least one of the following types of data:
a synthetic target medical image by applying a first trained AI-based model to the raw projection medical image or
a synthetic raw projection medical image by applying a second trained AI-based model to the target medical image,
first representation data representing a flavor of the target medical image by applying a third trained AI-based model to the target medical image.

Hence, the generation unit is used for replacing missed matching image data.

In a preferred variant of the adaption device according to one or more example embodiments of the present invention, the estimation unit is arranged for generating an estimated medical image by applying the medical image processing chain to the raw projection medical image or the synthetic raw projection medical image. Advantageously, the estimation unit is capable to complete the incomplete matching pair of medical image data related to a target flavor such the an adaption of the medical image processing chain to a desired flavor can be successfully performed.

Also preferred, the comparison unit of the adaption device is arranged for
determining a result of a comparison between the estimated medical image and
the target medical image or
the synthetic target medical image or for
generating second representation data representing a flavor of the estimated medical image by applying the third trained AI-based model to the estimated medical image and for determining a result of a comparison between the first representation data and the second representation data.

Advantageously, the type of comparison depends on the type of comparable data, in particular it depends thereon if the content of the estimated medical image and the target medical image is the same or not. Advantageously, the comparison unit is enabled to compare even different medical images including different image contents.

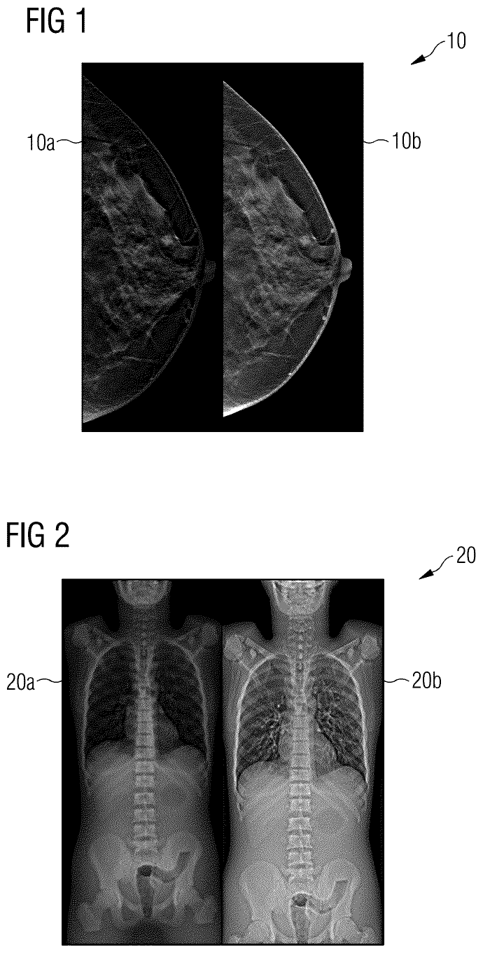

In FIG. 1, a schematic view 10 on two reconstructed volumes 10a, 10b of the same breast scan showing two different flavors is shown. As can be taken from FIG. 1, the left volume 10a is “darker” than the right volume 10b. The characteristic property of “flavour” can be defined based on a determined set of different parameters. The type of these parameters and hence the definition of the “flavour” is different dependent on the manufacturer of the X-ray imaging system.

In FIG. 2, a schematic view 20 on a scan of two different versions of images 20a, 20b of the same phantom generated by different medical imaging systems is shown. As can be taken from FIG. 2, the left volume 20a is “darker” than the right volume 20b.

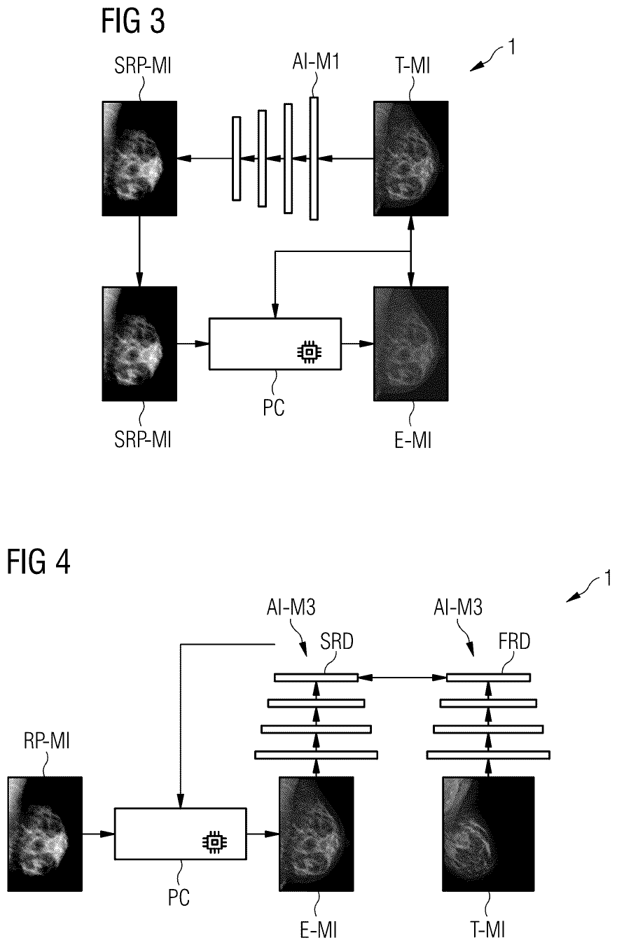

In FIG. 3, a schematic diagram 1 illustrating a method for AI-assisted generating an adapted medical image processing chain A-PC according to a first embodiment of the invention is shown.

A first artificial neural network AI-M1 can be trained to generate matching medical images SRP-MI, T-MI. This can be achieved, by estimating matching unprocessed synthetic raw projection medical images SRP-MI to the target medical images T-MI with the desired flavor. This case is used in the optimization depicted in FIG. 3.

However, it is also possible, to estimate processed images i.e. synthetic target medical images to corresponding raw projection medical images SRP-MI using a second artificial neural network AI-M2 (shown in FIG. 5). The generated synthetic target medical images should then have the same flavor as target medical images comprising the desired flavor. One way to train such a network are un- or self-supervised methods. This problem is often called “unpaired image-to-image translation” in literature. Having generated a matching pair of a raw projection medical image SRP-MI and an assigned target medical image T-MI, the medical image processing chain PC can be applied to the matching pair and an estimated medical image E-MI is generated based on the raw projection medical image SRP-MI of the matching pair. After that, a comparison between the estimated medical image E-MI and the target medical image T-MI is performed and the parameter values of the parameterized medical image processing chain PC are changed depending on the result of the comparison. The before-mentioned steps can be iteratively repeated, until an optimum of an adapted medical image processing chain PC, based on the result of the comparison, is achieved.

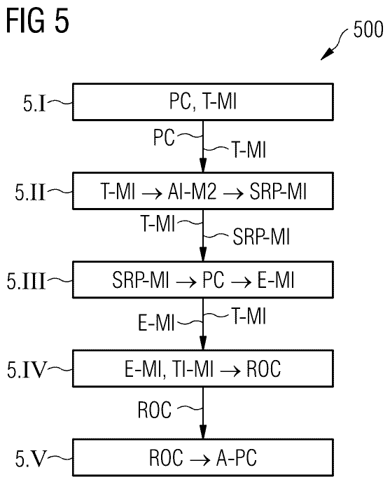

In FIG. 4, a schematic diagram 1 illustrating a method for AI-assisted generating an adapted medical image processing chain A-PC according to a second embodiment of the invention is depicted.

As in the first embodiment illustrated in FIG. 3, the medical image processing chain PC generates an estimated medical image E-MI based on a raw projection medical image RP-MI.

The idea underlying the second embodiment is that an objective function can be designed, which is able to compare the flavor of two medical images, even though these two medical images do not have the same content. A third AI-based model AI-M3 can be trained, which is able to represent the flavor of an estimated medical image E-MI or a target medical image T-MI without taking into account the different content of these images E-MI, T-MI. The representation data FRD, SRD related to the flavor of two medical images, i.e. the estimated medical image E-MI and the target medical image T-MI, can then be compared against each other. Hence, the generation of such representation data FRD, SRD enables the comparison of an estimated medical image E-MI, processed by the medical image processing chain PC, with a target medical image T-MI including the desired flavor and based thereon, an objective function can be generated, which can be used for an optimization of the medical image processing chain PC.

In FIG. 5, a flow chart diagram 500 illustrating the method for AI-assisted generating an adapted medical image processing chain A-PC according to the first embodiment of the invention is depicted.

In step 5.I, a medical image processing chain PC is received and a target medical image T-MI is received.

In step 5.II, a synthetic raw projection medical image SRP-MI which comprises the same content as the received target medical image T-MI is generated by applying a second trained AI-based model AI-M2 to the target medical image T-MI received in step 5.I.

In step 5.III, an estimated medical image E-MI is generated by applying the medical image processing chain PC to the synthetic raw projection medical image SRP-MI, generated in step 5. II.

In step 5.IV, a result ROC of a comparison between the estimated medical image E-MI and the target medical image T-MI received in substep 5.I is determined.

In step 5.V, an adapted medical image processing chain A-PC is generated by adapting the medical image processing chain PC based on the result ROC of the comparison.

It has to be mentioned that the steps 5.III to 5.V are iteratively repeated, until a quality criteria is achieved. The achievement of the quality criteria is determined based on the comparison in step 5.IV. That means that the iteration is repeated, until the precision of the optimization achieves a predetermined level or an optimum level.

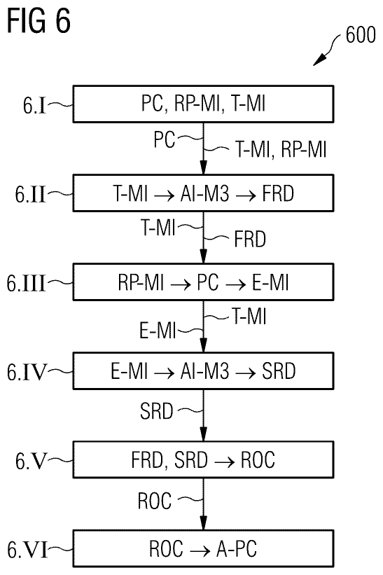

In FIG. 6, a flow chart diagram 600 illustrating the method for AI-assisted generating an adapted medical image processing chain A-PC according to a second embodiment of the invention corresponding to the scheme shown in FIG. 4, is presented.

In step 6.I, a medical image processing chain PC and a raw projection medical image RP-MI and a target medical image T-MI, wherein the content of the raw projection medical image RP-MI is not the same as the content of the target medical image T-MI, are received.

In step 6.II, first representation data FRD representing a flavor of the target medical image T-MI are generated by applying a third trained AI-based model AI-M3 to the target medical image T-MI received in Step 6.I.

In step 6.III, an estimated medical image E-MI is generated by applying the medical image processing chain PC to the raw projection medical image RP-MI received in step 6.I.

In step 6.IV, second representation data SRD representing a flavor of the estimated medical image E-MI are determined by applying the third trained AI-based model AI-M3 to the estimated medical image E-MI.

In step 6.V, a result ROC of a comparison between the first representation data FRD generated in step 6. II and the second representation data SRD is determined.

In step 6.VI, an adapted medical image processing chain A-PC is generated by adapting the medical image processing chain PC based on the result ROC of the comparison.

It has to be mentioned that the steps 6. III to 6.VI are iteratively repeated, until a quality criteria is achieved. The achievement of the quality criteria is determined based on the comparison in step 6.V.

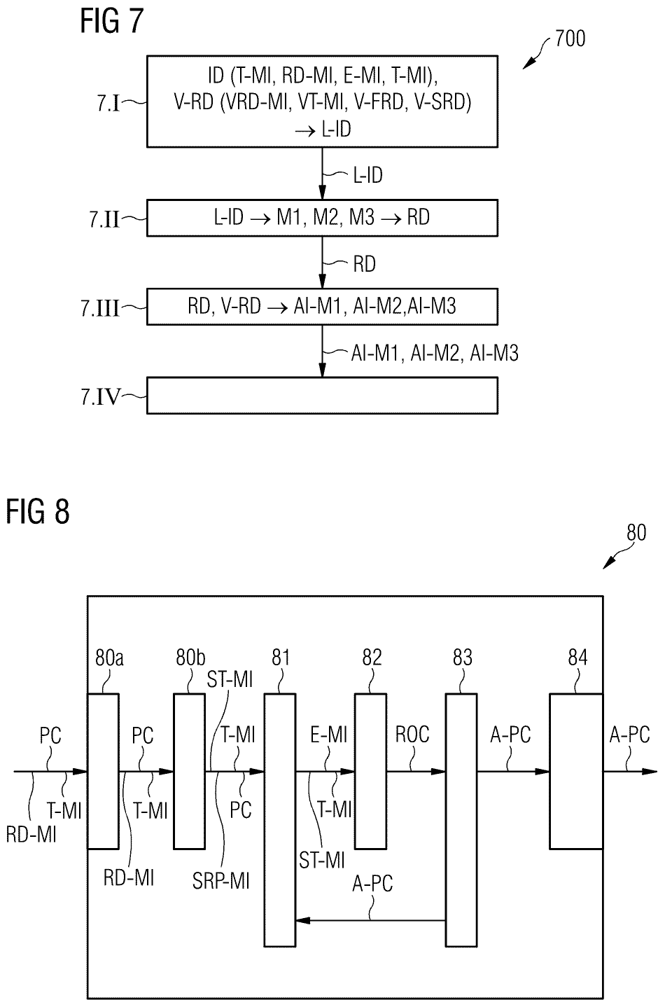

In FIG. 7, a flow chart diagram 700 illustrating the method for generating a trained AI-based model AI-M1, AI-M2, AI-M3 for estimating a missing piece of information of a complete matching pair of medical image data related to a target flavor, in particular synthetic medical images SRP-MI or representation data FRD, SRD, according to an embodiment of the invention, is depicted.

In step 7.I, labelled input data L-ID including input data ID and validated result data V-RD are generated. The labelled input data L-ID comprise one of the following data sets:
a target medical image T-MI as input data ID and a validated raw projection medical image VRP-MI as validated result data V-RD,
a raw projection medical image RP-MI as input data ID and a validated target medical image VT-MI as validated result data V-RD,
an estimated medical image E-MI or a target medical image T-MI as input data ID and validated representation data V-FRD, V-SRD representing a flavor of the medical image E-MI, T-MI as validated result data V-RD.

In step 7.II, the labelled input data L-ID are applied to an AI-based model M1, M2, M3 to be trained, wherein result data RD are generated.

In step 7.III, a trained AI-based model M1, M2, M3 is generated based on the result data RD and the validated result data V-RD.

In step 7.IV, the trained AI-based model AI-M1, AI-M2, AI-M3 is provided to a user.

In FIG. 8, a schematic view on an adaption device 80 according to an embodiment of the invention is shown.

The adaption device 80 comprises an input interface 80a for receiving a medical image processing chain PC and in the variant shown in FIG. 8, a target medical image T-MI or alternatively raw projection medical images RP-MI.

Further, the adaption device 80 includes a generation unit 80b for generating a synthetic raw projection medical image SRP-MI by applying a second trained AI-based model AI-M2 to the target medical image T-MI or alternatively synthetic target medical images ST-MI if only raw projection medical images RP-MI were received.

Part of the adaption device 80 is also an estimation unit 81 for generating an estimated medical image E-MI by applying the medical image processing chain PC to the synthetic raw projection medical image SRP-MI or alternatively to the raw projection medical image RP-MI.

The adaption device 80 also comprises a comparison unit 82 for determining a result ROC of a comparison between the estimated medical image E-MI and the target medical image T-MI or alternatively the synthetic target medical image ST-MI.

Further, the adaption device 80 includes an adaption unit 83 for generating an adapted medical image processing chain A-PC by adapting the medical image processing chain PC based on the result ROC of the comparison. In an iteration, the adaption unit 83 transmits the adapted medical image processing chain A-PC to the estimation unit 81 for generating an estimated medical image E-MI by applying the adapted medical image processing chain A-PC to the synthetic raw projection medical image SRP-MI or alternatively to the raw projection medical image RP-MI.

Furthermore, the adaption device 80 comprises an output interface 84 for outputting the adapted image processing chain A-PC after completing the iteration between the adaption device 83, the estimation unit 81 and the comparison unit 82.

The above descriptions are merely preferred embodiments of the present disclosure but not intended to limit the present disclosure, and any modifications, equivalent replacements, improvements, etc. made within the spirit and principle of the present disclosure should be included within the scope of protection of the present disclosure.

Further, the use of the undefined article “a” or “one” does not exclude that the referred features can also be present several times. Likewise, the term “unit” or “device” does not exclude that it consists of several components, which may also be spatially distributed. Furthermore, independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms s such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A method for AI-assisted generating an adapted medical image processing chain, the method comprising:
receiving medical image data representing only a piece of information of a complete matching pair of medical image data related to a target flavor, wherein a missing piece of information of the complete matching pair is missing from the received medical image data;
generating an estimated medical image by applying the medical image processing chain to a raw projection medical image related to the received medical image data;
determining a result of a comparison based on the estimated medical image and a target medical image related to the received medical image data; and
generating an adapted medical image processing chain by adapting the medical image processing chain based on the result of the comparison,
wherein the missing piece of information is generated based on the received medical image data using an AI-based model.

2. The method of claim 1, wherein the generating the estimated model, the determining the result of the comparison and the generating the adapted medical image processing chain are automatically iteratively repeated using the adapted medical image processing chain as a subsequent medical image processing chain for the generating the estimated medical image.

3. The method of claim 1, wherein the generating the estimated model, the determining the result of the comparison and the generating the adapted medical image processing chain are automatically iteratively repeated, until a predetermined quality criteria or optimum criteria for the result of the comparison is achieved.

4. The method of claim 1, wherein
the received medical image data comprises a raw projection medical image,
the generation of the missing piece of information comprises the generation of a synthetic target medical image by applying a first trained AI-based model to the raw projection medical image, and
the determining the result of the comparison is performed by comparing the estimated medical image with the synthetic target medical image being used as the target medical image.

5. The method of claim 1, wherein
the received medical image data comprises a target medical image,
the generation of the missing piece of information comprises the generation of a synthetic raw projection medical image by applying a second trained AI-based model to the target medical image,
the generating the estimated model is performed by applying the medical image processing chain to the synthetic raw projection medical image being used as the raw projection medical image, and
the determining the result of the comparison is performed by comparing the estimated medical image with the target medical image.

6. The method of claim 1, wherein
the received medical image data comprise a raw projection medical image and a target medical image, wherein the content of the raw projection medical image is not the same as the content of the target medical image,
the generation of the missing piece of information comprises the generation of first representation data representing a flavor of the target medical image in advance by applying a third trained AI-based model to the target medical image, and
the determining the result of the comparison includes,
generating second representation data representing a flavor of the estimated medical image by applying the third trained AI-based model to the estimated medical image, and
determining the result by a comparison between the first representation data and the second representation data.

7. The method of claim 6, wherein
the first representation data and the second representation data comprise a unit vector, and
a number of dimensions of the unit vector is the same as a number of different possible flavors represented by the first representation data and the second representation data.

8. The method of claim 7, wherein the result of the comparison comprises a difference between a first unit vector of the first representation data and a second unit vector of the second representation data.

9. The method of claim 6, wherein the first representation data and the second representation data comprise several activated last layers of an AI-based network, and the AI-based network is based on contrastive learning or siamese learning.

10. A method for generating a trained AI-based model for estimating a missing piece of information, the method comprising:
generating input data, wherein the input data comprise medical image data representing only a piece of information of a complete matching pair of medical image data related to a target flavor,
applying the input data to an AI-based model to be trained, wherein result data are generated,
training the AI-based model based on the result data, and
providing the trained AI-based model.

11. The method of claim 10, wherein the input data comprise labelled input data, including input data and validated result data, wherein the labelled input data comprise one of the following data sets:
a target medical image as input data and a validated raw projection medical image as validated result data,
a raw projection medical image as input data and a validated target medical image as validated result data,
an estimated medical image or target medical image as input data and validated representation data representing the flavor of the medical image as validated result data, and
and the training the AI-based model comprises training based on the result data and the validated result data.

12. The method of claim 10, wherein the input data are generated as unlabelled input data and the training the AI-based model comprises an unsupervised or self-supervised training.

13. An adaption device, comprising:
an input interface configured to receive medical image data representing only a piece of information of a complete matching pair of medical image data related to a target flavor, wherein a missing piece of information of the complete matching pair is missing from the received medical image data;
an estimation unit configured to generate an estimated medical image by applying a medical image processing chain to a raw projection medical image related to the received medical image data;
a comparison unit configured to determine a result of a comparison based on the estimated medical image and a target medical image related to the received medical image data; and
an adaption unit configured to generate an adapted medical image processing chain by adapting the medical image processing chain based on the result of the comparison,
wherein the missing piece of information is generated based on the received medical image data using an AI-based model.

14. A computer program product comprising instructions which, when executed by a computer, cause the computer to perform the method of claim 1.

15. A non-transitory computer program comprising instructions which, when executed by a computer, cause the computer to perform the method of claim 1.

16. The method of claim 2, wherein
the received medical image data comprises a raw projection medical image,
the generation of the missing piece of information comprises the generation of a synthetic target medical image by applying a first trained AI-based model to the raw projection medical image, and
the determining the result of the comparison is performed by comparing the estimated medical image with the synthetic target medical image being used as the target medical image.

17. The method of claim 2, wherein
the received medical image data comprises a target medical image,
the generation of the missing piece of information comprises the generation of a synthetic raw projection medical image by applying a second trained AI-based model to the target medical image,
the generating the estimated model is performed by applying the medical image processing chain to the synthetic raw projection medical image being used as the raw projection medical image, and
the determining the result of the comparison is performed by comparing the estimated medical image with the target medical image.

18. The method of claim 2, wherein
the received medical image data comprise a raw projection medical image and a target medical image, wherein the content of the raw projection medical image is not the same as the content of the target medical image,
the generation of the missing piece of information comprises the generation of first representation data representing a flavor of the target medical image in advance by applying a third trained AI-based model to the target medical image, and
the determining the result of the comparison includes,
generating second representation data representing a flavor of the estimated medical image by applying the third trained AI-based model to the estimated medical image, and
determining the result by a comparison between the first representation data and the second representation data.
